(19) United States
(12) Patent Application Publication
(10) Pub. No.: US 2017/0326353 A1
(43) Pub. Date: Nov. 16, 2017

(54) ELECTRODE FOR ELECTROSTIMULATION

(71) Applicant: ANT APPLIED NEUROSCIENCE TECHNOLOGIES GMBH, Dresden (DE)

(72) Inventor: Thomas FISCHER, Dresden (DE)

(73) Assignee: ANT APPLIED NEUROSCIENCE TECHNOLOGIES GMBH, Dresden (DE)

(21) Appl. No.: 15/532,551

(22) PCT Filed: Dec. 4, 2015
(86) PCT No.: PCT/EP2015/078665

§ 371 Date: Jun. 2, 2017

(30) Foreign Application Priority Data
Dec. 4, 2014 (DE) 10 2014 117 908.0

(51) Int. Cl. A61N 1/04

(52) U.S. Cl. CPC A61N 1/0492; A61N 1/0452; A61N 1/0456

(57) ABSTRACT

An electrode for electrostimulation includes an electric path having a storage structure for an electrolyte which comprises a front side for placing the electrode onto the skin of a living being and a rear side for making electric contact, at least one electrically conductive contacting structure, and an electric pad comprising at least one barrier structure for preventing a lateral flow of electrolytes.

The invention relates to an electrode for electrostimulation in medical and non-medical applications, for example for measuring or introducing currents into muscles or nerves, comprising an elastic pad having a storage structure for an electrolyte, which comprises a front side for placing the electrode onto the skin of a living being and a rear side for making electric contact, and at least one electrically conductive contacting structure.

Electrodes of the aforementioned type for the electrostimulation of muscles or nerves, as well as for measuring or introducing currents in humans and animals, are generally known. The electrodes are usually provided either with fixed cables or have connections such as sockets or the like for connecting cables for connecting the electrodes to measuring or therapy devices.

For transmitting currents between the electrode and the skin, the electrodes each comprise a conductive, usually metallic contact surface, on which an elastic pad having a storage capacity for a liquid or gel-like electrolyte is attached. In order to ensure a defined electrically conductive connection, the elastic pad is soaked with an electrolyte such as isotonic saline or the like prior to use.

DE 10 2007 046 886 A1 discloses a planar electrode pad to be brought in contact with the human body, by means of which current pulses are to be transmitted for the stimulation of muscles. Since this electrode pad is worn over sportswear and thus does not lie directly on the skin, the pad should have a liquid reservoir which under pressure is wetting the worn sportswear to ensure the flow of the current pulses.

An electrode for medical use is known from U.S. Pat. No. 4,092,985 A, the electrode consisting of a metal layer disposed between a cover layer and a fabric layer. Also from each of U.S. Pat. No. 4,852,572 A and U.S. Pat. No. 5,337,748 A an electrode for the medical field of application is known, wherein the electrodes are applied to a base layer and are covered by a gel layer.

The invention is based on the object of providing an electrode for electrostimulation, for example for medical applications, such as measuring or introducing currents into muscles or nerves, in particular for transcranial electrostimulation, but also for non-medical applications, for example, in the field of cognition research, which, on the one hand, allows for uniform contacting over the entire electrode surface, and at the same time reduces or prevents the undesired leakage of electrolyte from the elastic pad.

This object is achieved by an electrode of the type mentioned above, wherein the elastic pad comprises at least one barrier structure for preventing a lateral flow of electrolyte. Advantageous embodiments and further developments are described in the dependent claims.

In an electrode for electrostimulation, comprising an elastic pad having a storage structure for an electrolyte which comprises a front side for placing the electrode onto the skin of a living being and a rear side for making electric contact, and further comprising at least one electrically conductive contacting structure, it is proposed that the elastic pad has at least one barrier structure for preventing a lateral flow of electrolyte.

The term “lateral” is intended to mean a flow of the electrolyte in the plane of the electrode, i.e. parallel to the front side of the elastic pad which comes into contact with the skin, which should not occur as far as possible. In other words, the elastic pad is provided with structures which prevent or at least strongly hinder the unimpeded flow in this plane, so that the electrolyte remains in place for an even contact of the skin.

According to a first embodiment it can be provided that the elastic pad is segmented into at least two subareas by at least one barrier structure. In this way, several contact surfaces to the skin are provided in parallel which ensure uniform contact per se and together. It can be further provided that each subarea of the elastic pad comprises its own electrically conductive contacting structure. For example, for this purpose, a bump, which can be electrically contacted separately, can be arranged on the rear side of the elastic pad on each subarea.

Alternatively, it can be provided that all subareas of the elastic pad comprise a common electrically conductive contacting structure. This can be realized, for example, by the rear side of the elastic pad being electrically conductive in its entirety or an additional electrically conductive contact surface being connected or electrically contacted with the rear side of the elastic pad, as will be explained in more detail below.

According to a further embodiment, it is provided that a barrier structure encloses the edge region of the elastic pad. In this way it can be achieved that the electrolyte is not squeezed out of the pad laterally because the barrier structure proposed here forms a circumferential lateral boundary.

According to a further embodiment, it is provided that at least one barrier structure comprises a groove-like depression between two adjacent subareas. In this embodiment, the groove-like depression between two adjacent subareas acts as a barrier structure, which prevents lateral electrolyte flow between these subareas.

According to a further embodiment, it is provided that the elastic pad is made from a foamed elastomer. For example, the storage structure can be formed from open-pore foamed elastomer and at least one barrier structure can comprise a region of the elastic pad which is formed from a non-foamed elastomer or closed-pore foamed elastomer.

For example, the elastic pad may be made from a selectively opened foamed elastomer, wherein the storage structure is made of foamed open-pore elastomer, and at least one barrier structure comprises a region of the elastic pad formed from a foamed closed-pore elastomer.

Foamed elastomer is typically closed-pored after manufacture. Closed-pore elastomer is well suited as a barrier structure to prevent lateral flow of electrolyte. The elastomer, on the other hand, must be open-pored for the storage structure which is intended to receive the electrolyte. The pores in the regions of the elastic pad provided as a storage structure can be opened by selective perforation or compression. With a perforating or pressing tool, which only affects the storage structure, i.e., optionally provided subareas of the elastic pad, and thereby opens its pores, which itself comprises segments, for example, which define the shape, arrangement and size of the subareas, a simple segmentation of the elastic pad can be effected in which selectively only subareas of the elastic pad are opened by the segments of the perforating or pressing tool by perforation or pressure.

Alternatively, the elastic pad may be made of a selectively foamed elastomer, wherein the storage structure is formed from foamed elastomer and at least one barrier structure comprises a region of the elastic pad formed from non-foamed elastomer. Here, too, the pores are to be opened by means of a targeted action after the foaming of the elastomer, to the extent that the elastomer forms a closed-pore foam during foaming.

The term “selectively foamed” is intended to mean that at least one subarea of the front side of the elastic pad facing the skin is formed from foamed material, an open-pore foam being preferred, and other subareas are not foamed. The non-foamed subareas then form barrier structures which prevent the lateral flow of electrolyte between adjacent subareas or, in the case of a barrier structure enclosing the elastic pad in the edge region, the lateral leakage of electrolyte. This embodiment can be combined with the previously described embodiment, i.e., for example, it may be provided that each subarea is enclosed by a strip of non-foamed elastomer or closed-pore foamed elastomer and additionally a groove-like depression is arranged between the subareas.

If the elastomer is closed-pored after foaming, the electrical conductivity between the rear side and the front side of the elastic pad can also be achieved by subsequently compressing the foamed areas or simply or preferably several times perforating them so that the same effect is achieved as with an open-pore foam, namely that from the front side to the rear a continuous connection of the electrolyte stored in the material is achieved.

The regions in which barrier structures are to be produced, that is to say, for example, the circumferential edge region of the elastic pad and/or strip-shaped regions which segment the front side of the elastic pad into two or more subareas, can be generated by just not foaming the elastomer in these regions so that the elastomer remains relatively compact and thus liquid-impermeable compared to the other foamed regions, wherein the foamed regions serve the accommodation and storage of electrolyte and thus the electrical contacting of the skin.

According to a further embodiment, it is provided that the elastic pad has a continuous rear side layer of non-foamed elastomer on the rear side. In doing so, only the elastic pad itself must be produced for producing the electrode because the electrolyte can not penetrate to the rear side of the elastic pad.

According to a further embodiment, it is provided that at least one barrier structure is formed from several of at least partially interconnected material strands arranged one above the other. The material strands can, for example, consist of a non-foamed material such as silicone or the like. They can be generated, for example, by means of a plotter. If several such material strands are stacked one on top of the other, they form together a barrier structure which has approximately the shape of a wall, wherein material strands arranged one of top of the other can be connected to one another over their entire length or section-wise. Even if the material strands are not connected to one another over their entire length, they form a barrier structure in the sense of the technical teachings disclosed herein because they strongly hinder the electrolyte from flowing in the plane of the electrode, i.e., in parallel to the front side of the elastic pad that is in contact with the skin.

Furthermore, it can be provided that at least two intersecting barrier structures are formed in that material strands of a first barrier structure and material strands of a second barrier structure intersecting the first barrier structure are arranged alternately one above the other, wherein mutually contacting material strands of the first barrier structure and the second barrier structure are connected to one another in the intersection region. For example, a plotter can first produce material strands in several rows. Subsequently, material strands are produced in several columns, so that the material strands lying in columns intersect the material strands lying in rows and rest thereon. This is followed by a layer in rows followed by a layer in columns, etc. Mutually intersecting walls are formed that prevent a lateral flow of electrolyte, wherein the material strands in rows connect with the material strands in columns in the respective region where they mutually intersect, before curing of the material strands.

In addition, it can be provided that the the elastic pad comprises a rear side layer of mutually intersecting material strands on the rear side, which are connected to each other in the intersection region. The thus produced rear side layer can be used to attach the elastic pad and/or to the electrical contacting. The rear side layer and the barrier structures can be produced, for example, in one and the same operation by first placing material stands with a very narrow spacing in rows and then also with very narrow spacing in columns which combine to form a planar structure which is either closed (if the material strands have a sufficiently narrow spacing) or which comprises small holes between the material strands similar to a planar fabric. Subsequently, the barrier structures can be produced on this planar structure, which forms the rear side layer of the elastic pad, as described above, without having to interrupt the flow of material.

For electrical contacting, the elastic pad can comprise on its rear side one or more, for example, one for each subarea delimited by barrier structures, electrical contacting structures, for example a bump to which an electrical cable can be connected. In other words, in this embodiment, an additional electrically conductive connection can be dispensed with. However, such an additional electrically conductive contact surface can also advantageously be provided in this embodiment.

According to a further embodiment it can be provided that the elastomer is electrically conductive at least in the region of the storage structure. At the same time, it can be provided that the elastomer is electrically non-conductive at least in the region of the barrier structure. This further improves the electrical conductivity of the elastic pad in conjunction with the electrolyte stored therein. The electrically nonconductive design of the barrier structures, on the other hand, prevents a short circuit between the individual subareas of the storage structure, so that it is measurable and recognizable for each individual area whether there is sufficient electrical contact with the skin. The electric conductivity of the elastomer can be achieved, for example, by admixing electrically conductive particles, for example metal or graphite, with the elastomer.

In particular, in cooperation with a continuous rear side layer made from foamed elastomer arranged on the rear side of the elastic pad, the configuration involving an electrically conductive elastomer results in that an electrical conductivity of the elastic pads of high homogeneity can be achieved.

In addition, it can be provided that the elastic pad comprises a circumferential elastic collar for securing the elastic pad to the electrically conductive contacting structure. In this case, for example, a metal plate constitutes a base plate of the electrically conductive contacting structure, which is contacted by means of cables, while the elastic pad, on the one hand, is in electrically conductive connection with the electrically conductive contact surface and, on the other hand, makes contact with the skin.

In this case, the elastic pad assumes only mediation between the electrically conductive contact surface and the skin, and the elastic pad can in this case act as a disposable consumable material, while the additional electrically conductive contact surface can be reused. This also meets high hygienic requirements.

In a further embodiment, it can be provided that, on the additional electrically conductive contact surface at least one contacting structure is arranged that projects into the elastic pad. As a result, the electrical contact between the additional electrically conductive contact surface and the elastic pad is significantly improved. If the elastic pad is segmented in subareas, it is appropriate to provide, for each individual subarea, a contact element projecting into the respective subarea of the elastic pad which, for example, can have the shape of a cone which penetrates the rear side of the elastic pad when the elastic pad is applied by light pressure.

The invention is described below in detail with reference to exemplary embodiments and associated drawings. Here

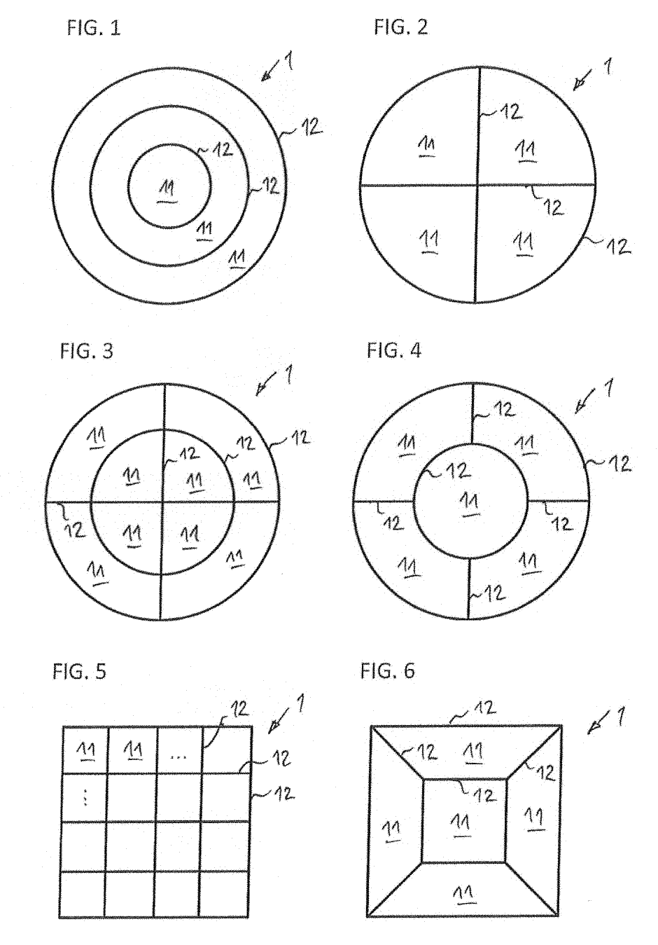

FIGS. 1 to 6 show plan views of exemplary embodiments of elastic pads with several subareas and barrier structures,

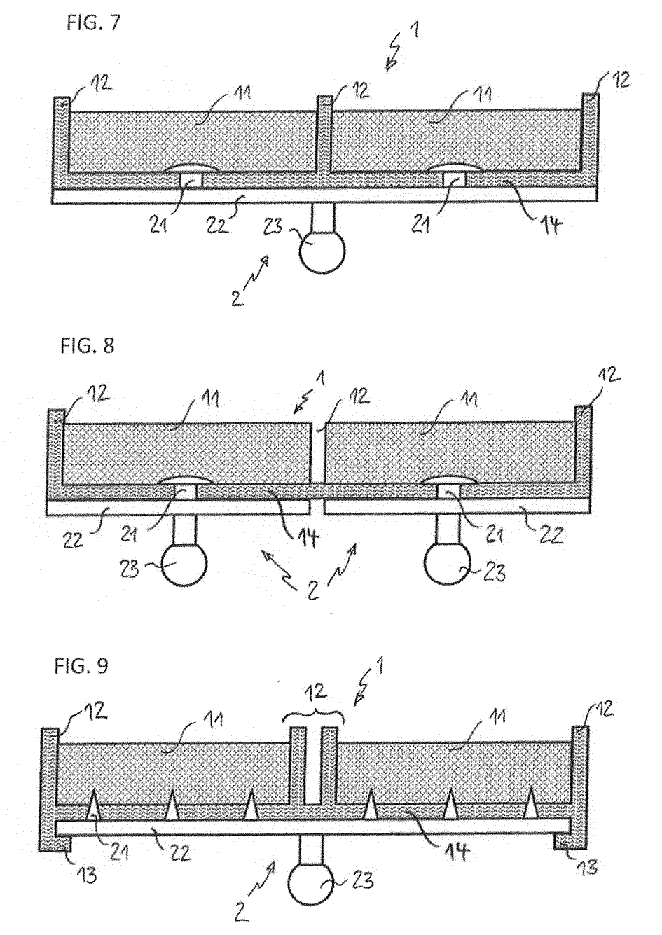

FIGS. 7 to 9 show cross-sectional views of exemplary electrodes, and

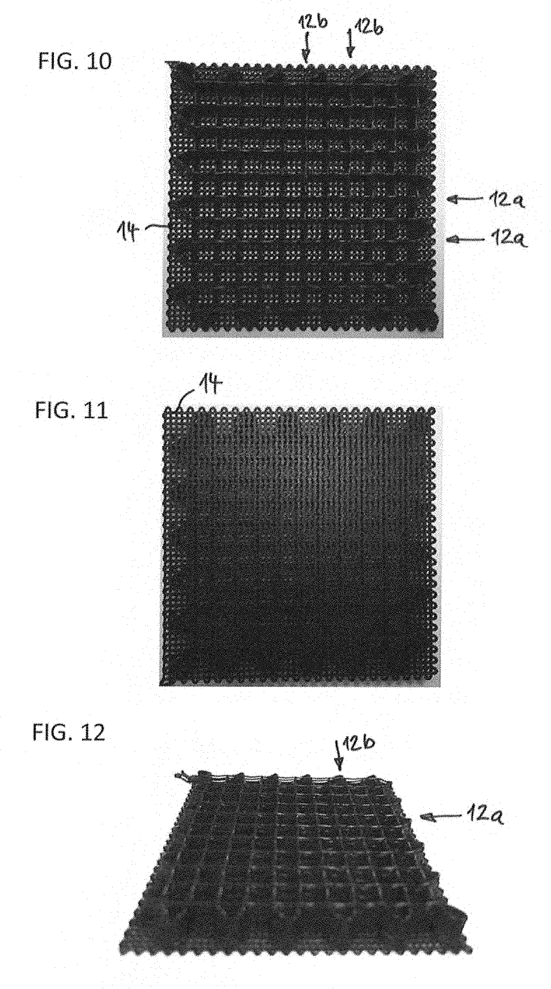

FIGS. 10 to 12 show three views of an exemplary embodiment.

The illustrations of FIGS. 1 to 6 show various embodiments of elastic pads 1 comprising barrier structures 12 for hindering a lateral flow of electrolyte, wherein these barrier structures 12 are only schematically indicated as dashes, without going into their specific configuration in more detail.

Each of the elastic pads 1 shown is subdivided into several subareas 11 by such barrier structures 12, whereby barrier structures 12 arranged between two adjacent subareas 11 prevent the lateral flow of electrolyte between these subareas 11.

In addition, a barrier structure 12 is arranged in each case in such a way that it encloses the edge region of elastic pad 1 so that the lateral leakage of electrolyte from elastic pad 1 is also prevented.

In the exemplary embodiments of FIGS. 1 to 4, elastic pads 1 each have a circular shape, whereas elastic pads 1 are square in the exemplary embodiments of FIGS. 5 and 6. Other forms, for example, oval or polygonal basic shapes of elastic pads 1 are also possible and are also encompassed by the invention.

The circular basic shape of elastic pad 1 is segmented into several subareas 11 in the exemplary embodiment of FIG. 1 by barrier structures 12 in the form of concentric circles, in the exemplary embodiment of FIG. 2 in the form of two perpendicular diameters, and in the exemplary embodiments of FIGS. 3 and 4, by a combination of concentric circles and radial lines.

In the exemplary embodiment of FIG. 5, the square basic shape of elastic pad 1 is divided into a matrix-like arrangement of square subareas 11 by barrier structures 12 in the form of lateral-parallel lines, while subareas 11 in the exemplary embodiment of FIG. 6 are produced by a combination of barrier structures 12 in the form of a square and several diagonally extending lines.

Specific embodiments of the barrier structures 12 as well as of the electrically conductive contacting structures are illustrated in the exemplary embodiments of FIGS. 7 to 9.

Shown in each case are cross-sections through the electrode, which comprises in each case an elastic pad 1 and an electrically conductive contacting structure 2.

Elastic pad 1 comprises in each case several subareas 11, two of which are visible in the selected sectional view. These subareas 11 are each delimited from one another by barrier structures 12 in such a way that a lateral flow of electrolyte flow, i.e., an exchange of electrolyte, is prevented between adjacent subareas 11. In addition, the edge regions of elastic pad 1 are also delimited by a circumferential barrier structure 12, so that a lateral leakage of electrolyte from elastic pad 1 is prevented.

Elastic pad 1 comprises a storage structure for an electrolyte. This storage structure corresponds to subareas 11, which are formed by an open-pore elastomeric foam. In the chosen illustration of FIGS. 7 to 9, the upper side of the electrode represents the surface which is brought into contact with the skin.

On the rear of elastic pad 1, i.e. in the chosen illustration at the bottom, there is a continuous rear side layer 14 made of closed-pore elastomeric foam. Elastic pad 1 is attached at this bottom portion in each case to a contacting structure 2. This contacting structure 2 is in electrically conductive connection with the storage structure. To this end, contacting structure 2 in each case comprises at least one base plate having at least one contact element 21 which establishes the conductive connection to a subarea 11, and at least one connection pin 23 for connecting a signal cable.

In the exemplary embodiment of FIG. 7, two contact elements 21, which establish the conductive connection to a respective subarea 11, are arranged on a base plate 22. All barrier structures 12 of elastic pad 1, that is to say both the barrier structure 12 enclosing elastic pad 1 and the barrier structure 12 which is arranged between the two subareas 11, like the rear side layer 14 of the elastic pad 1 consist of closed-pore elastomer foam.

In the embodiment of FIG. 8, contacting structure 2, on the other hand, comprises two base plates 22 each having one contact element 21, each contact element 21 establishing the conductive connection to one of the two subareas 11. Barrier structure 12 enclosing elastic pad 1, in turn, consists of non-closed-pored elastomeric foam, while the barrier structure 12 arranged between the two adjacent subareas 11 is formed by a groove-like depression between the adjacent subareas 11.

In the exemplary embodiment of FIG. 9, two contact elements 21 are arranged on a base plate 22 so that each contact element 21 establishes the conductive connection to one of the two subareas 11. In this exemplary embodiment, contact elements 21 are conical in shape, so that their tips can easily penetrate the rear side layer 14 of the electric pad which are made of non-foamed elastomer.

In this exemplary embodiment, the barrier structure 12 enclosing elastic pad 1 does not consist of foamed elastomer. The barrier structure 12 arranged between the two adjacent subareas 11, on the other hand, comprises a groove-like depression between the adjacent subareas 11 and, in addition, on the edge of each subarea, a strip of non-foamed elastomer.

In this exemplary embodiment, elastic pad 1 additionally comprises a circumferential elastic collar 13, with which elastic pad 1 is attached at the electrically conductive contacting structure 2.

All the barrier structures 12 in the exemplary embodiments of FIGS. 7 to 9, which consist of non-foamed elastomer are configured to be higher than the storage structures, i.e., the barrier structures 12 of non-foamed elastomer top the front side of elastic pad 1 and thus the skin facing surface of the storage structure.

The illustrations of FIGS. 10 to 12 show different views of an elastic pad 1, which consists entirely of plotted silicone strands, which form a rear side layer 14 as well as barrier structures 12 to hinder a lateral flow of electrolyte. FIG. 10 shows a plan view of the front side, FIG. 11 shows a plan view of the rear side, and FIG. 12 shows a perspective view.

Rear side layer 14 is formed of a layer of several rows and columns of mutually intersecting material strands, which are respectively connected in the intersection region, wherein the spacings between the material strands are large enough to leave small holes between them.

Barrier structures 12, which were also produced from mutually intersecting material strands in the same operation, are arranged on the rear side layer. In this case, material strands deposited in rows of several first barrier structures 12a and material strands deposited in columns of several second barrier structures 12b intersecting the first barrier structures 12a are arranged alternately one above the other, wherein material strands of the first storage structures 12a and the second storage structures 12b in the respective intersection region are connected to one another. The arrows indicate the direction in which the first barrier structures 12a and the second barrier structures 12b run.

The exemplary embodiment shown corresponds approximately to the configuration of FIG. 5. However, it is understood that using the method described any other configurations such as are shown, for example, in FIGS. 1 to 4 and 6, can be made.

### LIST OF REFERENCE NUMERALS

1 Elastic pad
11 Subarea
12 Barrier structure
12a First barrier structure
12b Second barrier structure
13 Collar
14 Rear side layer
2 Contacting structure
21 Contact element
22 Baseplate
23 Connection pin

1. An electrode for electrostimulation, comprising an elastic pad having a storage structure for an electrolyte, a front side for placing the electrode onto skin of a living being and a rear side for making electric contact, and at least one electrically conductive contacting structure, wherein said elastic pad comprises at least one barrier structure for preventing a lateral flow of electrolyte.

2. The electrode according to claim 1, wherein said elastic pad is segmented into at least two subareas by the at least one barrier structure.

3. The electrode according to claim 2, wherein each subarea of said elastic pad comprises its own electrically conductive contacting structure.

4. The electrode according to claim 2, wherein all subareas of said elastic pad comprise a common electrically conductive contacting structure.

5. The electrode according to claim 1, wherein the barrier structure encloses an edge region of said elastic pad.

6. The electrode according to claim 1, wherein the at least one barrier structure comprises a groove-like depression between two adjacent subareas.

7. The electrode according to claim 1, wherein said elastic pad comprises a foamed elastomer.

8. The electrode according to claim 7, wherein the storage structure comprises an open-pore foamed elastomer.

9. The electrode according to claim 7, wherein the at least one barrier structure comprises a region of said elastic pad of non-foamed elastomer.

10. The electrode according to claim 1, wherein the at least one barrier structure comprises several of at least partially interconnected material strands arranged one above the other.

11. The electrode according to claim 10, further comprising at least two mutually intersecting barrier structures formed in such a way that material strands of a first storage structure and material strands of a second storage structure intersecting said first storage structure in an intersecting region are arranged alternately one above the other, and wherein mutually contacting material strands of said first storage structure and said second storage structure are connected to one another in the intersection region.

12. The electrode according to claim 9, wherein regions of non-foamed elastomer of said elastic pad project beyond a surface of the storage structure forming the front side of said elastic pad.

13. The electrode according to claim 1, wherein said elastic pad comprises a continuous rear side layer of non-foamed elastomer or closed-pore foamed elastomer on the rear side.

14. The electrode according to claim 1, wherein said elastic pad on the rear side comprises a rear side layer of mutually intersecting material strands, which are connected to one another in an intersection region.

15. The electrode according to claim 1, wherein said storage structure is electrically conductive.

16. The electrode according to claim 1, wherein the at least one barrier structures is electrically non-conductive.

17. The electrode according to claim 1, wherein said elastic pad comprises a circumferential elastic collar for securing said elastic pad to said electrically conductive contacting structure.

18. The electrode according to claim 1, wherein said electrically conductive contacting structure comprises at least one contact element projecting into said elastic pad.

19. The electrode according to claim 7, wherein the at least one barrier structure comprises a region of said elastic pad of closed-pore foamed elastomer.

20. The electrode according to claim 19, wherein regions of closed-pore foamed elastomer of said elastic pad project beyond a surface of the storage structure forming the front side of said elastic pad.
